(19) United States
(12) Patent Application Publication
(10) Pub. No.: US 2009/0177134 A1
(43) Pub. Date: Jul. 9, 2009

(54) VACUUM HEAD DRESSING

(76) Inventor: Jake Timothy, Leeds (GB)

Correspondence Address: TAFT, STETTINIUS & HOLLISTER LLP, SUITE 1800, 425 WALNUT STREET, CINCINNATI, OH, 45202-3957, US

(21) Appl. No.: 12/281,676

(22) PCT Filed: Mar. 5, 2007
(86) PCT No.: PCT/GB07/00774

§ 371 Date: Nov. 18, 2008

(30) Foreign Application Priority Data
Mar. 6, 2006 (GB) 0604458.0

(51) Int. Cl. A61F 13/00

(52) U.S. Cl. 602/53

(57) ABSTRACT

A post-operative or trauma-site head dressing (10) comprises a cap (12) adapted to fit over the head (20) of a patient. The cap is a patchwork of compartments (14) of airtight, flexible material connected to a single opening (18) through which air may be evacuated. The compartments are filled with polystyrene beads so that, on air evacuation, the cap not only becomes rigid, but also gently presses against the scalp to inhibit post-operative swelling and/or bleeding from head wounds.

This invention relates to a method of, and apparatus for, reducing the occurrence of post-operative swelling in patient's undergoing surgery on the head, and in particular to a head dressing for such patients. The invention relates also to head dressings generally, for example to stem bleeding from the head of a victim at the site of an accident.

### BACKGROUND

Post-operative swelling is always an issue that is sought to be minimised. It hinders recovery. On the other hand, it cannot be eliminated altogether because the operation wound needs a blood supply to enable healing, and that inevitably results in some leakage, and hence swelling. Bandages are usually employed but, for head injuries, bandages are bulky and cumbersome. Indeed, most of the bandage employed does not come into contact with the wound but rather is around the head merely holding a small part of the bandage against the wound. Moreover, wounds need to be checked periodically, to ensure that there is not excessive leakage of fluid from the wound. Generally, bandages are disposed of when a wound is checked and this is wasteful.

U.S. Pat. No. 6,592,535 discloses apparatus for arresting scalp bleeding, and comprises a skull cap including a plurality of inflatable bladders each inflatable to press different parts of the head and effect haemostasis. Presumably, the device is intended for emergency situations in which stemming of bleeding from an open wound at an accident/incident site is desired. This is thus a temporary measure while the victim is awaiting proper medical attention. In this circumstance, the ability to inflate perhaps a single bladder that applies pressure only on the wound site is a satisfactory arrangement.

However, in a post operative situation, where a permanent dressing is needed, this single-site application of pressure would not be satisfactory.

My international application publication number WO-2005/097022 describes a post-operative head dressing comprising an expansion resistant cap adapted to fit over the head of a patient and a liner therefor, which liner comprises a network of tubes of resilient material connected to a single opening through which gas may be introduced to pressurise the liner network so that the liner expands against the cap to be urged inwardly, reducing its circumference about a periphery of the cap. When the cap is worn by a human patient and the liner is inflated, pressure is applied to the patient's scalp, at least around a periphery of the skull. This stems blood flow in the capillaries of the scalp and reduces inflammation therein.

Such an arrangement is satisfactory except that pressure points still develop and keeping the cap on the head is a problem.

It is known to employ pots and splints to set broken bones, or at least immobilise limbs in post-accident trauma situations. Such pots and splints take many forms and one form comprises an element having compartments formed from flexible material and filled with polystyrene or like beads. The flexible material is airtight and the compartments are provided with means to evacuate them of air. Once moulded around a patient's limb, the compartments are evacuated and atmospheric pressure compresses the beads inside the compartments into solid blocks, so that the element as a whole takes on a solid structure. In such a way, the limb becomes rigidly supported. The following patents disclose such or similar arrangements: GB-A-1422966, EP-A-189317, U.S. Pat. No. 6,308,353, U.S. Pat. No. 6,656,143. However, there is no suggestion that such an arrangement may have application as a bandage to stem bleeding or inhibit swelling.

Indeed, as in my previous application, it is an object of the present invention to provide an improved post-operative dressing that stems swelling and bleeding of a patient that has undergone surgery on their head. However, it is also an object of the present invention to provide a head dressing that can be employed at any time, for example, post accident trauma.

### BRIEF SUMMARY OF THE DISCLOSURE

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

In accordance with the present invention there is provided a head dressing comprising a cap formed by a flexible air-tight bag including a plurality of compartments filled with beads, the cap including means to evacuate the compartments whereby atmospheric pressure compresses the flexible material against the beads transforming them into a solid block. Preferably, the cap includes an adjustable temple band, whereby the band can be adjusted to fit around a wearer's head before the compartments are evacuated.

Surprisingly, it is found that not only does the cap become rigid, as one would have expected given the arrangements known in the art of bone splints using similar arrangements, but also that a gentle all-around pressure is exerted by the cap against the wearer's skull. This stems blood flow in the capillaries of the scalp and reduces inflammation therein, as well as bleeding from open wounds. Furthermore, because the element can be shaped against a wearer's skull by appropriate manipulation of the beads, the opportunities for pressure points, or, more particularly, absent-pressure points, are reduced. A more even application of pressure appears to be achieved.

Preferably, said compartments are interconnected so that said means to evacuate comprises a single opening. In this event, said bag is a single bag. On the other hand, there may be multiple bags, indeed, each with only a single compartment. Said bags may be quite independent, but may be separate except for a common conduit leading from the opening of each bag to a single means of evacuation for all the bags together. Preferably, said opening includes a non-return valve. Preferably, a hand pump is provided by means of which said evacuation may be achieved.

Preferably, said cap comprises an elongate compartment forming said temple band, the ends of which may be releasably connected together at variable positions, to one side of which band there is connected a patchwork of further compartments shaped to form a dome, said side at the ends of said band not being connected to said further compartments. Preferably, in use, said ends are arranged at a front of the cap, whereby they are connected together in the region of a wearer's forehead.

Preferably, said releasable connection is effected by hook and loop fastening material. The beads may conveniently be polystyrene, and of approximately round shape. However, the constituency or shape of the beads is not fundamental to the operation of the dressing provided that the beads do not slide over one another in a fluid-like manner once compressed together by atmospheric pressure.

In use, wounds will be covered with a gauze dressing, to prevent direct contact between the tubes and a patients' skin.

Different caps may be provided to enable more accurate fitting of the cap to people's (differently-shaped) heads.

The invention also provides method of reducing post-operative swelling of the head of a human or animal patient, or for controlling blood loss from wounds in the skull comprising the steps of:
a) applying to the head a cap formed by a flexible air-tight bag including a plurality of compartments filled with beads, the cap including means to evacuate the compartments; and,
b) evacuating the compartments whereby atmospheric pressure compresses the flexible material against the beads transforming them into a solid block.

Preferably, said cap employed in said method is the cap of a dressing as defined above.

### BRIEF DESCRIPTION OF THE DRAWINGS

An embodiment of the invention is further described hereinafter, by way of example, with reference to the accompanying drawings, in which:

FIG. 1 is a perspective view of a patient's head on which a cap in accordance with the present invention is fitted;

FIG. 2 is a front view of the cap of FIG. 1; and

FIG. 3 is a section through the cap of FIG. 1 along the line III-III in that Figure.

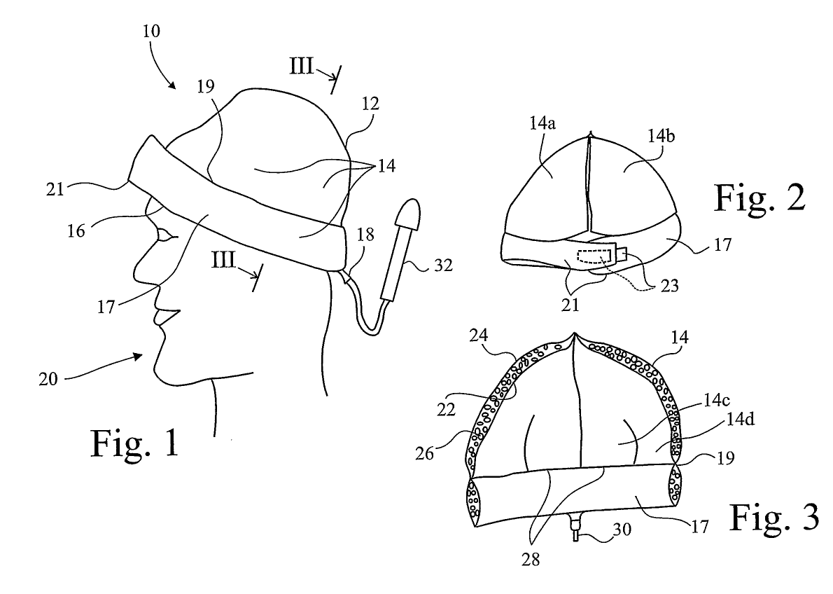

In FIG. 1, a head dressing 10 comprises a cap element 12 consisting of a patchwork of compartments 14 which are interconnected together in the form of a hemispherical dome, having a rim 16. Rim 16 is formed by a single band compartment 17. At the back of the rim 16 is a single port 18 through which air can be introduced to, and evacuated from, the dressing 10.

In use, a patient 20 has undergone surgery of one description or another on their head, or suffered a head trauma resulting in a wound. At the end of surgery, the wound (not shown) is closed in the normal way, and covered with breathable gauze (not shown). Alternatively, at an accident site the patient is assessed for suitability and need for a head dressing to stem blood loss from a head wound. Then, the cap 12 is placed over the head 20, enclosing the wound and its gauze covering. The liner 12 is a loose fit over the skull of the patient. Different sizes are made available for patients with differently sized heads. However, although band 17 is connected along most of one side 19 to the patchwork dome 14, at its ends 21 it is not connected to the dome and so a certain degree of adjustability is provided in the circumference of the cap. Thus, once placed on the head 20, hoop and hook patches 23 on the respective ends 21 of the band 17 are engaged so as to provide a snug-fit around the patient's head.

Patchwork 14, in its simplest form, is merely four triangular pieces 14a,b forming a pyramid. However, they may be Y-shaped, with arms 14c,d being joined to provide some roundness to the arrangement to better approximate the shape of the skull.

In any event, each compartment 14 is composed of inner layer 22 and outer layer 24 of airtight material sealed around all edges, so that the compartments so formed are airtight. Inside each compartment is disposed numerous polystyrene beads 26 approximately filling the space of the compartments, but not so much as to pressurise their inner and outer layers.

All the interiors of the compartments 14 are preferably linked by passages 28 formed by breaks in the connections between inner and outer layers 22,24 and between the adjacent compartments. Therefore it is necessary to provide only one air valve 18. This is preferably in the form of a switchable non-return valve and has a screw connection 30 to permit detachable connection of a suction pump 32.

When the cap 12 is fitted on the patient's head, and tabs 23 connected so that the band 17 is a snug fit, the compartments may be shaped by hand so that the beads inside conform to the shape of the patients skull. Then, once the pump 32 has been connected, the compartments 14 are evacuated so that the beads 26 are compressed together by atmospheric pressure and take on a solid construction. Furthermore, they tighten evenly onto the skull, applying an even pressure quite adequate to stem bleeding from an open wound, or indeed, to prevent swelling.

In addition, by virtue of the restriction of blood supply, swelling associated with the trauma of surgery can be limited.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A head dressing comprising a cap formed by a flexible air-tight bag including a plurality of compartments filled with beads, the cap including means to evacuate the compartments whereby atmospheric pressure compresses the flexible material against the beads transforming them into a solid block.

2. A dressing as claimed in claim 1, in which the cap includes an adjustable temple band, whereby the band can be adjusted to fit around a wearer's head before the compartments are evacuated.

3. A dressing as claimed in claim 2, in which said cap comprises an elongate compartment forming said temple band, the temple band having two ends which may be releasably connected together at variable positions, and to one side of which band there is connected a patchwork of further compartments shaped to form a dome, the ends of said band not being connected to said further compartments.

4. A dressing as claimed in claim 3, in which, in use, said ends are arranged at a front of the cap, whereby they are connected together in the region of a wearer's forehead.

5. A dressing as claimed in claim 3, in which a releasable connection is effected by hook and loop fastening material.

6. A dressing as claimed in claim 1, in which said compartments are interconnected so that said means to evacuate comprises a single opening.

7. A dressing as claimed in claim 1, in which there are multiple bags, each with only a single compartment.

8. A dressing as claimed in claim 7, in which said bags are quite independent, except for a common conduit leading from the opening of each bag to a single means of evacuation for all the bags together.

9. A dressing as claimed in claim 1, in which, said means to evacuate includes a non-return valve.

10. A dressing as claimed in claim 1, in which the means to evacuate the compartments is a hand pump.

11. A dressing as claimed in claim 1, in which the beads are polystyrene.

12. More than one of a dressing as claimed in claim 1, wherein each dressing is of a different size to enable more accurate fitting of the cap to people's heads.

13. A method of reducing post-operative swelling of the head of a human or animal patient, or for controlling blood loss from wounds in the skull comprising the steps of.
a) applying to the head a cap formed by a flexible air-tight bag including a plurality of compartments filled with beads, the cap including means to evacuate the compartments; and,
b) evacuating the compartments whereby atmospheric pressure compresses the flexible material against the beads transforming them into a solid block.

14. A method as claimed in claim 13, in which said cap employed in said method is the head dressing as claimed in claim 1.

15. (canceled)
